(19) United States
(12) Patent Application Publication
(10) Pub. No.: US 2026/0278782 A1
(43) Pub. Date: Sep. 17, 2026

(54) INFORMATION PROCESSING DEVICE, INFORMATION PROCESSING METHOD, AND RECORDING MEDIUM

(71) Applicant: NEC Corporation, Tokyo (JP)

(72) Inventors: Hirokazu SAKAMOTO, Tokyo (JP); Kenichi KAMIJO, Tokyo (JP)

(73) Assignee: NEC Corporation, Tokyo (JP)

(21) Appl. No.: 19/163,902

(22) PCT Filed: Mar. 24, 2023
(86) PCT No.: PCT/JP2023/011947

§ 371 Date: Sep. 10, 2025

(51) Int. Cl. G06T 7/00; G16H 30/20

(52) U.S. Cl. CPC G06T 7/0012; G16H 30/20; G06T 2207/10068; G06T 2207/30096

(57) ABSTRACT

An information processing device includes, at least one memory storing instructions, and at least one processor configured to execute the instructions to, acquire one or a plurality of endoscopic images, detect a polyp candidate from the endoscopic image, calculate a first type score indicating a polyp likelihood regarding the polyp candidate, store an image of the polyp candidate and the first type score in a storage unit, determine whether the polyp candidate is identical to one or a plurality of other polyp candidates stored in the storage unit; and calculate a first type integrated score by integrating the first type score and a first type score related to one or a plurality of the other polyp candidates stored in the storage unit when the candidates are determined identical to each other.

### TECHNICAL FIELD

The present invention relates to an information processing device, an information processing method, and an information processing program.

### BACKGROUND ART

Conventionally, there has been known a technique of supporting a medical worker by referring to a medical image such as an endoscopic image, estimating whether a disease exists in each site included in the image, the severity of the disease, or the like, and providing a medical worker with an estimation result.

For example, PTL 1 discloses an endoscopic image processing device that determines the severity (score) of inflammatory bowel disease from an endoscopic image.

### CITATION LIST

### Patent Literature

PTL 1: WO 2023/282144 A1

### SUMMARY OF INVENTION

### Technical Problem

In general, in the above-described technology, reliability is required for a probability value (estimated value) related to a disease. The technique described in PTL 1 has room for improvement from the viewpoint of providing a more reliable probability value.

An aspect of the present invention has been made in view of the above problems, and an object thereof is to provide a technology capable of providing a more reliable value as a probability value regarding a disease with reference to an endoscopic image.

### Solution to Problem

An information processing device according to one aspect of the present invention includes an acquisition means for acquiring one or a plurality of endoscopic images, a detection means for detecting a polyp candidate from the endoscopic image and calculating a first type score indicating a polyp likelihood regarding the polyp candidate, a storage control means for storing an image of the polyp candidate detected by the detection means and the first type score in a storage unit, a first determination means for determining whether the polyp candidate detected by the detection means is identical to one or a plurality of other polyp candidates stored in the storage unit, and an integration means for calculating a first type integrated score by integrating the first type score calculated by the detection means and a first type score related to one or a plurality of the other polyp candidates stored in the storage unit in a case where the first determination means determines that the candidates are identical to each other.

An information processing method according to one aspect of the present invention causes at least one processor to execute acquiring one or a plurality of endoscopic images, detecting a polyp candidate from the endoscopic image and calculating a first type score indicating a polyp likelihood regarding the polyp candidate, storing an image of the polyp candidate and the first type score in a storage unit, determining whether the polyp candidate is identical to one or a plurality of other polyp candidates stored in the storage unit, and calculating a first type integrated score by integrating the first type score and a first type score related to one or a plurality of the other polyp candidates stored in the storage unit in a case where the candidates are determined to be identical to each other.

An information processing program according to one aspect of the present invention causes a computer to execute an acquisition process for acquiring one or a plurality of endoscopic images, a detection process for detecting a polyp candidate from the endoscopic image and calculating a first type score indicating a polyp likelihood regarding the polyp candidate, a storage control process for storing an image of the polyp candidate detected by the detection process and the first type score in a storage unit, a first determination process for determining whether the polyp candidate detected by the detection process is identical to one or a plurality of other polyp candidates stored in the storage unit, and an integration process for calculating a first type integrated score by integrating the first type score calculated by the detection process and a first type score related to one or a plurality of the other polyp candidates stored in the storage unit in a case where the first determination process determines that the candidates are identical to each other.

### Advantageous Effects of Invention

According to one aspect of the present invention, a more reliable value can be provided as a probability value regarding a disease with reference to an endoscopic image.

### BRIEF DESCRIPTION OF DRAWINGS

FIG. 1 is a block diagram illustrating a configuration of an information processing device according to a first example embodiment.

FIG. 2 is a flowchart illustrating a flow of an information processing method according to the first example embodiment.

FIG. 3 is a block diagram illustrating a configuration of an information processing device according to a second example embodiment.

FIG. 4 is a diagram for explaining processing by the information processing device according to the second example embodiment.

FIG. 5 is a flowchart illustrating a flow of processing by the information processing device according to the second example embodiment.

FIG. 6 is a diagram illustrating an example of a display image generated by an information processing device according to the second example embodiment.

FIG. 7 is a diagram illustrating an example of a display image generated by the information processing device according to the second example embodiment.

FIG. 8 is a diagram illustrating an example of a display image generated by the information processing device according to the second example embodiment.

FIG. 9 is a diagram illustrating an example of a display image generated by the information processing device according to the second example embodiment.

FIG. 10 is a diagram illustrating an example of a display image generated by the information processing device according to the second example embodiment.

FIG. 11 is a block diagram illustrating a configuration of a computer that functions as the information processing device according to each example embodiment.

### EXAMPLE EMBODIMENT

### First Example Embodiment

A first example embodiment of the present invention will be described in detail with reference to the drawings. The present example embodiment is a basic form of the example embodiment described below.

### <Outline of Information Processing Device 1>

Schematically describing one aspect of an information processing device 1 according to the present example embodiment, the information processing device 1 detects a disease candidate together with a score (probability value, estimated value) indicating disease likelihood from an input endoscopic image, and calculates an integrated score by integrating scores regarding a plurality of detected disease candidates.

In the following description, an example of an endoscopic image obtained from an endoscope inserted into a digestive tract such as a large intestine will be described as an example of the endoscopic image, but this does not limit the present example embodiment. Examples of the object to be detected with reference to the endoscopic image include a polyp candidate and the presence or absence of inflammation, but the present example embodiment is not limited thereto.

### <Configuration of Information Processing Device 1>

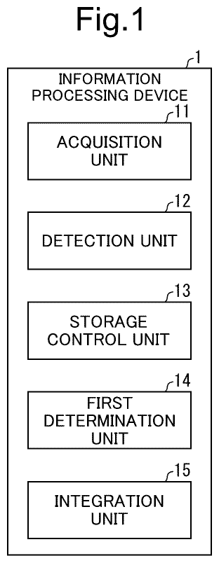

Hereinafter, the configuration of the information processing device 1 will be described with reference to FIG. 1. FIG. 1 is a block diagram illustrating a configuration of the information processing device 1. As illustrated in FIG. 1, the information processing device 1 includes an acquisition unit 11, a detection unit 12, a storage control unit 13, a first determination unit 14, and an integration unit 15.

### (Acquisition Unit 11)

The acquisition unit 11 acquires one or a plurality of endoscopic images. As an example, the acquisition unit 11 may acquire an endoscopic video including a series of a plurality of still images and acquire one or a plurality of still images included in the endoscopic video as the one or plurality of endoscopic images. In the following description, a case where the acquisition unit 11 acquires an endoscopic video from the digestive tract of the same subject and acquires one or a plurality of endoscopic videos from the endoscopic image will be considered. However, this is not limited to the present example embodiment. In a case where there are a plurality of subjects, it is possible to identify and manage an endoscopic image of a certain subject and an endoscopic image of another subject by giving a subject ID and the like to the endoscopic video.

### (Detection Unit 12)

The detection unit 12 detects a polyp candidate from the endoscopic image acquired by the acquisition unit 11 and calculates a first type score indicating the likelihood of a polyp related to the polyp candidate. Here, how to set the numerical range of the first score is not limited to the present example embodiment, but as an example, the first type score may be calculated as a numerical value equal to or more than 0 and equal to or less than 100, such as 70% and 80%, or the first type score may be calculated as a numerical value equal to or more than 0 and equal to or less than 1, such as 0.7 and 0.8.

The detection unit 12 may calculate one score as the first type score for one polyp candidate, or may calculate a plurality of scores. In the latter case, as an example, the detection unit 12 may separately calculate, for a certain polyp candidate, a first type score for whether the polyp candidate is an A-type polyp and a first type score for whether the polyp candidate is a B-type polyp.

The detection unit 12 may calculate a score regarding inflammation and a score regarding the possibility of another disease with reference to the endoscopic image acquired by the acquisition unit 11, and include these calculated scores in the first type score.

As the detection unit 12, a learned neural network or the like obtained by performing learning with reference to a plurality of learning images can be used, but this does not limit the present example embodiment.

### (Storage Control Unit 13)

The storage control unit 13 stores the image of the polyp candidate detected by the detection unit 12 and the first type score in a storage unit. Here, as an example, the storage unit may be a storage unit included in the information processing device 1, or may be a storage unit that exists separately from the information processing device 1 and is able to communicate with the information processing device 1. In a case where there are a plurality of subjects, the image of the polyp candidate and the first type score may be stored in the storage unit together with a subject ID for identifying the subject.

### (First Determination Unit 14)

The first determination unit 14 determines whether the polyp candidate detected by the detection unit 12 is identical to one or a plurality of other polyp candidates stored in the storage unit. Here, as an example, the determination of the identity by the first determination unit 14 is performed by comparing the image of the polyp candidate detected by the detection unit 12 with each image of one or a plurality of other polyp candidates stored in the storage unit, but this does not limit the present example embodiment.

The first determination unit 14 may determine the identity by further referring to other information in addition to the image. As an example, the acquisition unit 11 of the information processing device 1 may acquire position information regarding the position of the distal end portion of the endoscope together with the endoscopic image, and the first determination unit 14 may refer to the information regarding the position in the determination of the identity.

As the first determination unit 14, a learned neural network or the like obtained by performing learning with reference to a plurality of learning images can be used, but this does not limit the present example embodiment.

In the above description, the polyp candidate detected by the detection unit 12 and one or a plurality of other polyp candidates stored in the storage unit are described as targets to be compared by the first determination unit 14, but this does not limit the present example embodiment. As an example, the first determination unit 14 can compare the polyp candidate detected by the detection unit 12 and temporarily stored in the storage unit with one or a plurality of other polyp candidates stored in the storage unit. In other words, an image once stored in the storage unit may be set as a comparison target, or an image before being stored in the storage unit may be set as a comparison target.

A target to be compared by the first determination unit 14 is, for example, a polyp candidate related to the same subject. In a case where there are a plurality of subjects, the first determination unit 14 can refer to a subject ID associated with a polyp candidate and specify a polyp candidate related to the same subject.

### (Integration Unit 15)

In a case where the first determination unit 14 determines that the candidates are identical to each other, the integration unit 15 calculates a first type integrated score obtained by integrating the first type score calculated by the detection unit 12 and the first type score related to one or a plurality of the other polyp candidates stored in the storage unit. Here, a specific method of calculating the first type integrated score by the integration unit 15 is not limited to the present example embodiment, but the first type integrated score may be calculated by a simple average, a weighted average, or the like as an example.

As an example, in a case where the first type score calculated by the detection unit 12 for a certain polyp candidate is 90%, the first score for another polyp candidate stored in the storage unit is 80%, and the first type integrated score is calculated from these two scores, the first integrated score=85% may be calculated by simple averaging as follows:

$$( 90 + 80 ) / 2 = 85 .$$

As another example, in a case where the first type score calculated by the detection unit 12 for a certain polyp candidate is 90%, the first score for another polyp candidate stored in the storage unit is 80%, the weighting factor for the certain polyp is 0.6, and the weighting factor for the other polyp is 0,4, and in a case where the first type integrated score is calculated from these two scores and two weighting factors, the first integrated score=86% may be calculated by the weighted average as follows:

$$( 90 * 0.6 + 80 * 0.4 ) = 86 .$$

In a case where the weighted average is used in this manner, the weighting factor is set to have a positive correlation with indexes:
Inflammation score estimated from endoscopic image including the polyp candidate; and
Size of polyp candidate in endoscopic image including the polyp candidate. Here, the inflammation score is a score indicating the degree of inflammation at a site included in the endoscopic image, and can be calculated by the detection unit 12 as an example.

As described above, in the information processing device 1 according to the present example embodiment, a configuration is adopted in which one or a plurality of endoscopic images are acquired, a polyp candidate is detected from the endoscopic images, a first type score indicating a polyp likelihood regarding the polyp candidate is calculated, an image of the detected polyp candidate and the first type score are stored in a storage unit, whether the detected polyp candidate is identical to one or a plurality of other polyp candidates stored in the storage unit is determined, and the first type integrated score is calculated by integrating the calculated first type score and the first type score regarding one or a plurality of the other polyp candidates stored in the storage unit when it is determined that the detected polyp candidate and the one or a plurality of other polyp candidates are the same.

According to the information processing device 1 configured as described above, since the first integrated score is calculated from the first score of each of the plurality of polyp candidates determined to be the same, there is an effect that the reliability of the calculated score is improved.

### <Flow of Information Processing Method S1>

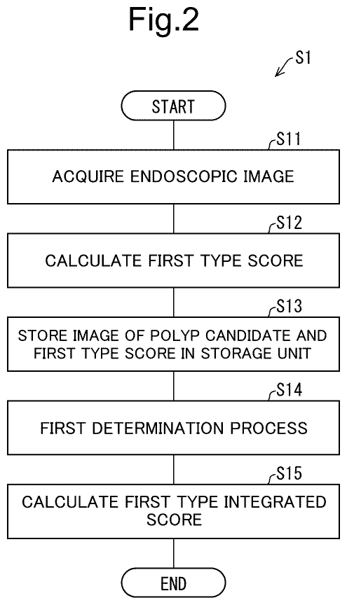

Next, a flow of an information processing method S1 according to the present first example embodiment will be described with reference to FIG. 2. FIG. 2 is a flowchart illustrating the flow of the information processing method S1. As illustrated in FIG. 2, in step S11, the acquisition unit 11 acquires one or a plurality of endoscopic images. Subsequently, in step S12, the detection unit 12 detects a polyp candidate from the endoscopic image and calculates a first type score indicating a polyp likelihood regarding the polyp candidate. Specific processing by the detection unit 12 is as described above.

Subsequently, in step S13, the storage control unit 13 stores the image of the polyp candidate detected by the detection unit 12 and the first type score in a storage unit. Subsequently, in step S14, the first determination unit 14 determines whether the polyp candidate detected by the detection unit 12 is identical to one or a plurality of other polyp candidates stored in the storage unit. Specific processing by the first determination unit 14 is as described above. Subsequently, in step S15, in a case where the first determination unit 14 determines that the candidates are identical to each other, the integration unit 15 calculates a first type integrated score obtained by integrating the first type score calculated by the detection unit 12 and the first type score related to one or a plurality of the other polyp candidates stored in the storage unit. Specific processing by the integration unit 15 is as described above.

According to the information processing method S1 configured as described above, similarly to the information processing device 1 described above, since the first integrated score is calculated from the first score of each of the plurality of polyp candidates determined to be the same, there is an effect that the reliability of the score calculated with reference to the endoscopic image is improved.

### Second Example Embodiment

A second example embodiment of the present invention will be described in detail with reference to the drawings. Components having the same functions as the components described in the first example embodiment are denoted by the same reference signs, and the description thereof will be omitted.

### <Configuration of Information Processing Device 1A>

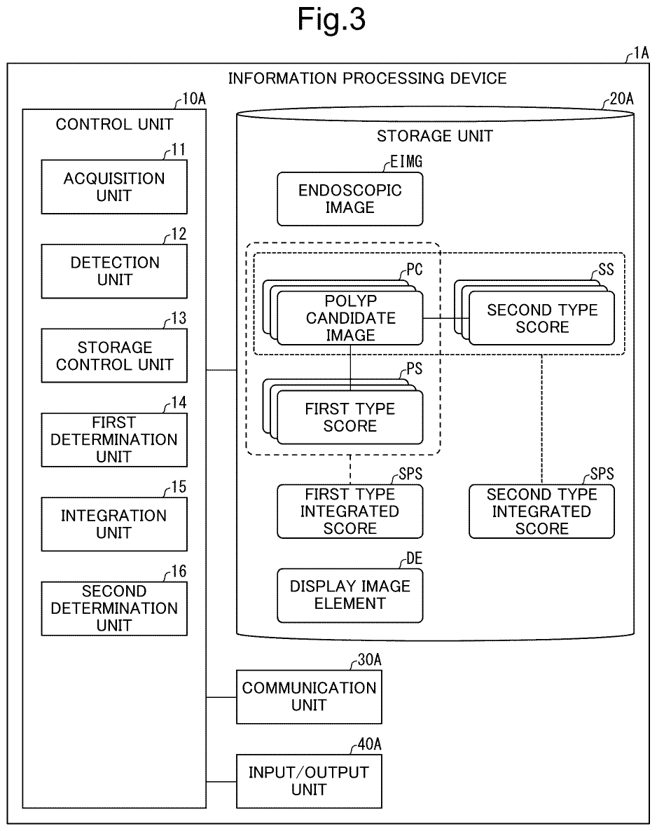

FIG. 3 is a block diagram illustrating the configuration of the information processing device 1A according to the present example embodiment. The information processing device 1A includes a control unit 10A, a storage unit 20A, a communication unit 30A, and an input/output unit 40A.

### (Communication Unit 30A)

The communication unit 30A communicates with a device outside the information processing device 1A via a communication line. Although a specific configuration of the communication line is not limited to the present example embodiment, the communication line is, for example, a wireless local area network (LAN), a wired LAN, a wide area network (WAN), a public line network, a mobile data communication network, or a combination thereof. The communication unit 30A transmits data supplied from the control unit 10A to another device, and supplies data received from another device to the control unit 10A.

### (Input/Output Unit 40A)

Input/output devices such as a keyboard, a mouse, a display, a printer, and a touch panel are connected to the input/output unit 40A. The input/output unit 40A accepts inputs of various types of information with respect to the information processing device 1A from the connected input device. The input/output unit 40A outputs various types of information to a connected output device under the control of the control unit 10A. Examples of the input/output unit 40A include an interface such as, for example, a universal serial bus (USB).

### (Acquisition Unit 11)

The acquisition unit 11 acquires one or a plurality of endoscopic images. As an example, the acquisition unit 11 may acquire an endoscopic video including a series of a plurality of still images and acquire one or a plurality of still images included in the endoscopic video as the one or plurality of endoscopic images. In the following description, similarly to the first example embodiment, a case where the acquisition unit 11 acquires an endoscopic video from the digestive tract of the same subject and acquires one or a plurality of endoscopic videos from the endoscopic image will be considered. However, this is not limited to the present example embodiment. In a case where there are a plurality of subjects, it is possible to identify and manage an endoscopic image of a certain subject and an endoscopic image of another subject by giving a subject ID and the like to the endoscopic video.

### (Detection Unit 12)

The detection unit 12 detects a polyp candidate from the endoscopic image acquired by the acquisition unit 11 and calculates a first type score indicating the likelihood of a polyp related to the polyp candidate. Here, how to set the numerical range of the first score is not limited to the present example embodiment, but as an example, the first type score may be calculated as a numerical value equal to or more than 0 and equal to or less than 100, such as 70% and 80%, or the first type score may be calculated as a numerical value equal to or more than 0 and equal to or less than 1, such as 0.7 and 0.8.

The detection unit 12 may calculate one score as the first type score for one polyp candidate, or may calculate a plurality of scores. In the latter case, as an example, the detection unit 12 may separately calculate, for a certain polyp candidate, a first type score for whether the polyp candidate is an A-type polyp and a first type score for whether the polyp candidate is a B-type polyp.

The detection unit 12 may refer to the endoscopic image acquired by the acquisition unit 11 and further calculate a second type score (also referred to as inflammation score) indicating the degree of inflammation.

As the detection unit 12, a learned neural network or the like obtained by performing learning with reference to a plurality of learning images can be used.

As an example, the detection unit 12 may include the following detection engine including a neural network:
(A-1) Polyp candidate detection engine for normal region (non-inflammatory region);
(A-2) Polyp candidate detection engine for an inflammatory region;
(B) Inflammation detection engine that determines whether it is an inflammatory region (outputs a score for inflammation), or the like. As an example, the detection engine may execute:
(1) Calculating a score related to inflammation by applying an inflammation detection engine (B) to the endoscopic image acquired by the acquisition unit 11;
(2) Calculating, when the score related to inflammation is less than a predetermined value, the first type score by applying the polyp candidate detection engine (A-1) for a normal region to the endoscopic image; and
(3) Calculating, when the score related to inflammation is equal to or more than a predetermined value, the first type score by applying the polyp candidate detection engine (A-2) for an inflammatory region to the endoscopic image.

The timing at which the detection unit 12 starts the process of detecting a polyp candidate is not limited to the present example embodiment, but may be a timing having consistency with a series of operations by a medical worker.

As an example, the detection unit 12 includes configurations as follows:
Starting detection of a polyp candidate when a detection start instruction from a medical worker is input to the input/output unit 40A included in the information processing device 1A; and
Starting detection of a polyp candidate when the control unit 10A detects that a predetermined site is included in the endoscopic image acquired by the acquisition unit 11. In the latter example, the control unit 10A may include a detection means for detecting whether the endoscopic image includes the image of the ileocecal valve, and the detection may be used as the trigger. In many cases, the polyp candidate is detected not at the time of insertion into the digestive tract but at the time of withdrawal from the digestive tract, and thus the above-described configuration is suitable.

### (Storage Control Unit 13)

The storage control unit 13 stores the image of the polyp candidate detected by the detection unit 12 and the first type score in a storage unit. The storage control unit 13 causes the storage unit to store the second type score calculated by the detection unit 12.

Here, the storage control unit 13 may cause the storage unit to store the second type score and the endoscopic image (image of a polyp candidate) referred to by the detection unit 12 to calculate the second type score in association with each other.

As an example, the storage unit may be a storage unit included in the information processing device 1, or may be a storage unit that exists separately from the information processing device 1 and is able to communicate with the information processing device 1. In a case where there are a plurality of subjects, the image of the polyp candidate and the first type score may be stored in the storage unit together with a subject ID for identifying the subject.

### (First Determination Unit 14)

The first determination unit 14 determines whether the polyp candidate detected by the detection unit 12 is identical to one or a plurality of other polyp candidates stored in the storage unit. Here, as an example, the determination of the identity by the first determination unit 14 is performed by comparing the image of the polyp candidate detected by the detection unit 12 with each image of one or a plurality of other polyp candidates stored in the storage unit, but this does not limit the present example embodiment.

As the first determination unit 14, a learned neural network or the like obtained by performing learning with reference to a plurality of learning images can be used, but this does not limit the present example embodiment.

In the above description, the polyp candidate detected by the detection unit 12 and one or a plurality of other polyp candidates stored in the storage unit are described as targets to be compared by the first determination unit 14, but this does not limit the present example embodiment. As an example, the first determination unit 14 can compare the polyp candidate detected by the detection unit 12 and temporarily stored in the storage unit with one or a plurality of other polyp candidates stored in the storage unit. In other words, an image once stored in the storage unit may be set as a comparison target, or an image before being stored in the storage unit may be set as a comparison target.

A target to be compared by the first determination unit 14 is, for example, a polyp candidate related to the same subject. In a case where there are a plurality of subjects, the first determination unit 14 can refer to a subject ID associated with a polyp candidate and specify a polyp candidate related to the same subject.

The first determination unit 14 may determine the identity by further referring to other information in addition to the image. As an example, the acquisition unit 11 of the information processing device 1 may acquire position information regarding the position of the distal end portion of the endoscope together with the endoscopic image, and the first determination unit 14 may refer to the information regarding the position in the determination of the identity.

Here, a specific example of the position information acquired by the acquisition unit 11 does not limit the present example embodiment, but may have, as an example, the following configurations:
A sensor capable of detecting a three-dimensional shape of an endoscope is included in the endoscope, and the acquisition unit 11 of the information processing device 1A acquires information regarding the shape, and position information obtained by the control unit 10A estimating the position of the distal end of the endoscope with reference to the acquired information is used; and
The position information obtained by estimating the position of the distal end of the endoscope by the control unit 10A with reference to the image in the digestive tract included in the endoscopic image is used.

Here, in the case of the latter example, as an example, a neural network learned using learning data in which an image in the digestive tract is associated with a position of a site in the digestive tract included in the image may be used for the estimation. As another example, an optical flow may be calculated from a series of a plurality of images in the digestive tract, a movement amount of the position of the distal end of the endoscope may be estimated based on the optical flow, and the position of the distal end of the endoscope may be estimated from the estimated movement amount.

### (Integration Unit 15)

In a case where the first determination unit 14 determines that the candidates are identical to each other, the integration unit 15 calculates a first type integrated score obtained by integrating the first type score calculated by the detection unit 12 and the first type score related to one or a plurality of the other polyp candidates stored in the storage unit. Here, a specific method of calculating the first type integrated score by the integration unit 15 is not limited to the present example embodiment, but the first type integrated score may be calculated by, as an example, the following methods:
Performing simple averaging;
Performing weighted averaging;
Extracting median; and
Extracting maximum value.

As an example, in a case where the first type score calculated by the detection unit 12 for a certain polyp candidate is 90%, the first score for another polyp candidate stored in the storage unit is 80%, and the first type integrated score is calculated from these two scores, the first integrated score=85% may be calculated by simple averaging as follows:

$$( 90 + 80 ) / 2 = 85 .$$

As another example, in a case where the first type score calculated by the detection unit 12 for a certain polyp candidate is 90%, the first score for another polyp candidate stored in the storage unit is 80%, the weighting factor for the certain polyp is 0.6, and the weighting factor for the other polyp is 0,4, and in a case where the first type integrated score is calculated from these two scores and two weighting factors, the first integrated score=86% may be calculated by the weighted average as follows:

$$( 90 * 0.6 + 80 * 0.4 ) = 86 .$$

In a case where the weighted average is used in this manner, the weighting factor is set to have a positive correlation with indexes:
Inflammation score estimated from endoscopic image including the polyp candidate;
Time during which the polyp candidate has been imaged as an endoscopic image (in other words, the number of frames of the endoscopic image including the polyp candidate); and
Size of polyp candidate in endoscopic image including the polyp candidate.

The integration unit 15 may calculate a second type integrated score by integrating the second type score calculated by the detection unit 12 and the second type score regarding one or a plurality of other endoscopic images stored in the storage unit.

Here, a specific example of the process of calculating the second type integrated score is not limited to the present example embodiment, but the second type integrated score may be calculated, similarly to the calculation of the first type integrated score described above, by the following methods:
Performing simple averaging;
Performing weighted averaging;
Extracting median; and
Extracting maximum value.

In a case where the detection unit 12 detects a plurality of inflammatory regions in the digestive tract, the detection unit 12 may assign identification numbers to these inflammatory regions, and the integration unit 15 may calculate the second type integrated score in the inflammatory regions to which the same identification number is assigned. For example, the integration unit 15 may calculate the second type integrated score regarding an inflammatory region 1 by integrating the second type scores associated with the plurality of endoscopic images obtained by imaging the inflammatory region 1, and calculate the second type integrated score regarding an inflammatory region 2 by integrating the second type scores associated with the plurality of endoscopic images obtained by imaging the inflammatory region 2.

As will be described later, the integration unit 15 may include a display image generation means (display image generation unit) for generating a display image. The integration unit 15 may include a display element determination means (display element determination unit) for determining a display element to be included in the display image.

According to the integration unit 15 configured as described above, since the first type integrated score is calculated by integrating the first type scores related to a plurality of polyp candidates determined to be the same, it is possible to provide a highly reliable score related to the polyp likelihood. Since the second type integrated score is calculated by integrating the plurality of second type scores, it is possible to provide a highly reliable score regarding the degree of inflammation.

### (Second Determination Unit 16)

A second determination unit 16 refers to the first type integrated score calculated by the integration unit 15 for a certain polyp candidate and makes a determination related to the certain polyp candidate.

Here, the specific determination processing by the second determination unit 16 is not limited to the present example embodiment, but as an example, the second determination unit 16 may determine whether the polyp candidate is a polyp with reference to the first type integrated score. For example, the second determination unit 16 may compare the first type integrated score with a predetermined threshold and determine that the polyp candidate is a polyp when the first type integrated score is equal to or more than the threshold. Here, the description of “determination” includes a concept similar to “estimation” in terms of information processing. The determination result by the second determination unit 16 is stored in the storage unit 20A as an example.

### (Flow of Processing by Information Processing Device 1A)

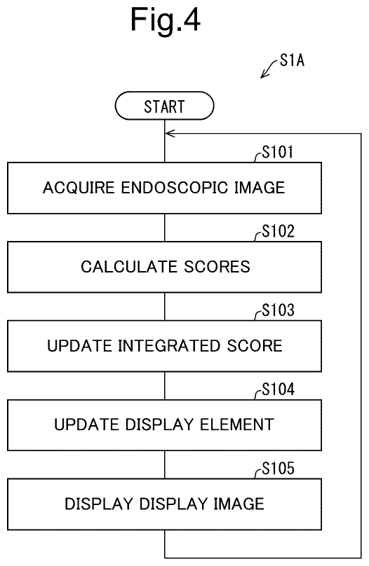

Hereinafter, a flow of processing executed by the information processing device 1A will be described with reference to FIG. 4. FIG. 4 is a flowchart illustrating a flow of processing executed by the information processing device 1A.

### (S101)

In step S101, the acquisition unit 11 acquires an endoscopic image. A specific processing example by the acquisition unit 11 is as described above.

### (S102)

Subsequently, in step S102, the detection unit 12 refers to the endoscopic image and calculates the first type score and the second type score described above. The manner of calculating these scores is as described above. This step can include each processing performed by the storage control unit 13 and the first determination unit 14 described above.

### (S103)

Subsequently, in step S103, the integration unit 15 calculates the above-described first type integrated score and the second type integrated score.

### (S104)

Subsequently, in step S104, the integration unit 15 updates the display element to be included in the display image according to the information obtained in the above step.

### (S105)

Subsequently, in step S105, the integration unit 15 causes the display unit to display the display image updated in step S104. As an example, a display panel functioning as the display unit may be included in the input/output unit 40A or may be communicable with the information processing device 1A via the input/output unit 40A.

After step S105 ends, the information processing device 1A returns to step S101 and repeats the processing.

(Processing example by information processing device 1A)

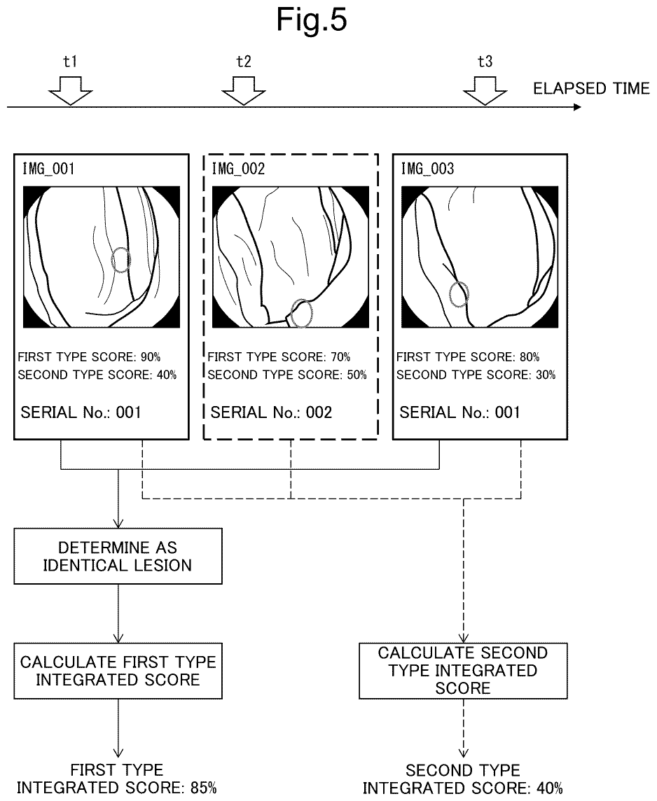

Hereinafter, a specific processing example by the information processing device 1A will be described with reference to FIG. 5. FIG. 5 is a diagram for explaining a processing example of an information processing device 1A.

FIG. 5 illustrates a case where the acquisition unit 11 acquires an endoscopic image IMG_001 at timing t1, acquires an endoscopic image IMG_002 at timing t2 which is timing after timing t1, and acquires an endoscopic image IMG_003 at timing t3 which is timing after timing t3.

FIG. 5 illustrates an example in which the detection unit 12 calculates each score as follows with reference to each of the above-described endoscopic images.
Endoscopic image IMG_001: First type score: 90%, Second type score: 40%
Endoscopic image IMG_002: First type score: 70%, Second type score: 50%
Endoscopic image IMG_003: First type score: 80%, Second type score: 30%

In the example of FIG. 5, the first determination unit 14 determines that a polyp candidate included in the endoscopic image IMG_001 is identical to a polyp candidate included in the endoscopic image IMG_003, and performs simple averaging to calculate:

(90+80)/2=85%,
as a first type integrated score regarding the polyp candidates determined to be the same.

As illustrated in FIG. 5, the first determination unit assigns the same serial number (serial number: 001 in the example of FIG. 5) to an endoscopic image (image of a polyp candidate) determined to be the same.

In the example illustrated in FIG. 5, the integration unit 15 performs simple averaging to calculate:

$$( 40 + 50 + 30 ) / 3 = 40 \% ,$$

as a second type integrated score from the endoscopic images IMG_001, IMG_002, and IMG_003.

As illustrated in FIG. 5, the integration unit 15 may calculate the second type integrated score with reference to all of the series of endoscopic images acquired by the acquisition unit 11, or may calculate the second type integrated score with reference to images obtained by thinning out the series of endoscopic images acquired by the acquisition unit 11.

### (Specific Example of Display Image)

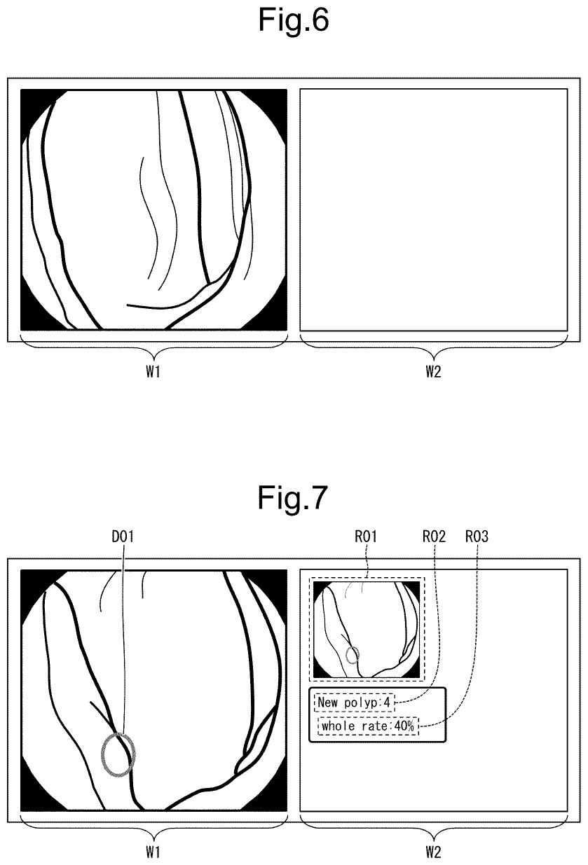

Hereinafter, an example of the display image generated by the integration unit 15 will be described with reference to FIGS. 6 to 10. FIGS. 6 to 10 are diagrams illustrating examples of display images generated by the integration unit 15.

FIG. 6 is a view illustrating a display image before a polyp candidate is detected at the time of inspection using the information processing device 1A. As illustrated in FIG. 6, the display image includes, as an example, a region W1 for displaying an endoscopic image and a region W2 for displaying various types of information and images obtained from the endoscopic image.

FIG. 7 illustrates an example of the display image generated by the integration unit 15 in a case where:
a polyp candidate D01 is detected by the detection unit 12 in the endoscopic image acquired by the acquisition unit 11; and
the first determination unit 14 determines that the polyp candidate D01 is not identical to any of the polyp candidates stored in the storage unit 20A. As illustrated in FIG. 7, the integration unit 15 disposes an endoscopic image (image of the polyp candidate D01) including the polyp candidate D01 in a region R01 in the region W2 in the display image. The integration unit 15 disposes a display element “New Polyp: 4” indicating that the polyp candidate D01 is a polyp that has not been detected so far and a serial number thereof is 4 in a region R02 in the region W2, and disposes a display element “whole rate: 40%” indicating that the second type integrated score (integrated inflammation score) is 40% in a region R03.

In this manner, the integration unit 15 may include the calculated second type integrated score in the display image. According to this configuration, the calculated second type integrated score (inflammation score after integration) can be presented in an easy-to-understand manner.

The integration unit 15 may include the second type integrated score in the display image when the detection unit 12 calculates the second type score a predetermined number of times or more. According to this configuration, it is possible to present the second type integrated score with higher reliability.

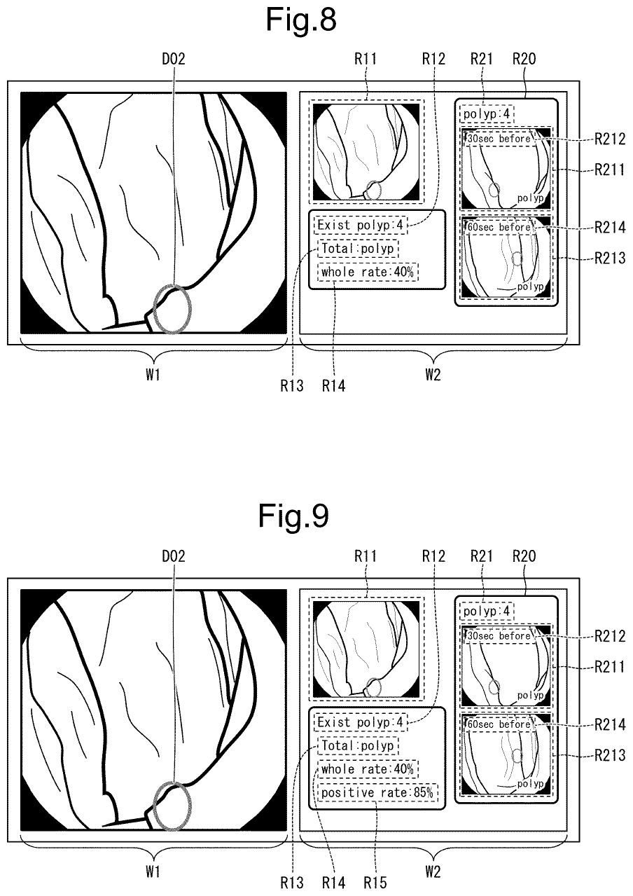

FIG. 8 illustrates an example of the display image generated by the integration unit 15 in a case where:
a polyp candidate D02 is detected by the detection unit 12 in the endoscopic image acquired by the acquisition unit 11; and
the first determination unit 14 determines that the polyp candidate D02 is identical to the polyp candidate stored in the storage unit 20A. As illustrated in FIG. 8, the integration unit 15 disposes an endoscopic image (image of the polyp candidate D02) including the polyp candidate D02 in a region R11 in the region W2 in the display image. The integration unit 15 disposes, in a region R12 in the region W2, a display element “Exist Polyp: 4” indicating that the polyp candidate D02 is a polyp detected so far and its serial number is 4. The integration unit 15 disposes, in the region R13, a display element “Total: polyp” indicating that the second determination unit 16 determines (estimates) that the polyp candidate D02 is a polyp, and disposes, in the region R14, a display element “whole rate: 40%” indicating that the second type integrated score (integrated inflammation score) is 40%.

As illustrated in FIG. 8, the integration unit 15 displays an image or the like of one or a plurality of polyp candidates determined to be the same as the polyp candidate D02, which are the detected polyp candidates stored in the storage unit, in a region R20 in the region W2.

In the example illustrated in FIG. 8, a display element “polyp: 4” indicating a serial number of these polyp candidates determined to be the same is disposed in a region R21 in the region R20, and each image of a polyp candidate determined to be the same as the polyp candidate D02, which is an image of the polyp candidate stored in the storage unit, is disposed in the region R211 and the region R213.

In a region R212, a display element “30 sec before” indicating that the image of the polyp candidate shown in a region R211 has been acquired 30 seconds before is disposed, and in a region R214, a display element “60 sec before” indicating that the image of the polyp candidate shown in the region R213 has been acquired 60 seconds before is disposed.

As illustrated in FIG. 8, in the information processing device 1A, in a case where the first determination unit 14 determines that the candidates are identical to each other, the integration unit 15 includes, in the display image, the endoscopic image acquired by the acquisition unit 11 (in this example, the image including the polyp candidate D02) and the images of one or a plurality of the other polyp candidates (in this example, images disposed in the regions R211 and R213) stored in the storage unit. By configuring the information processing device 1A as described above, the detected polyp candidate and the polyp candidate determined to be the same as the polyp candidate can be presented in an easy-to-understand manner.

As illustrated in FIG. 8, in the information processing device 1A, the integration unit 15 may include a serial number attached to the polyp candidate in the display image in a case where the first determination unit 14 determines that the polyp candidates are identical to each other. According to the information processing device 1A configured as described above, it is possible to present the polyp candidates determined to be the same in an easy-to-understand manner.

The example illustrated in FIG. 9 is a modification of the display image illustrated in FIG. 8. In the display image illustrated in FIG. 9, the first type integrated score calculated by the integration unit 15 is displayed in a region R15 in addition to the display elements illustrated in FIG. 8. In other words, the integration unit 15 may include the first type integrated score in the display image. In this manner, the integration unit 15 of the information processing device 1A may include the first type integrated score calculated by the integration unit 15 in the display image in addition to or instead of the second type integrated score calculated by the integration unit 15. According to the information processing device 1A configured as described above, the calculated first integrated score can be presented in an easy-to-understand manner.

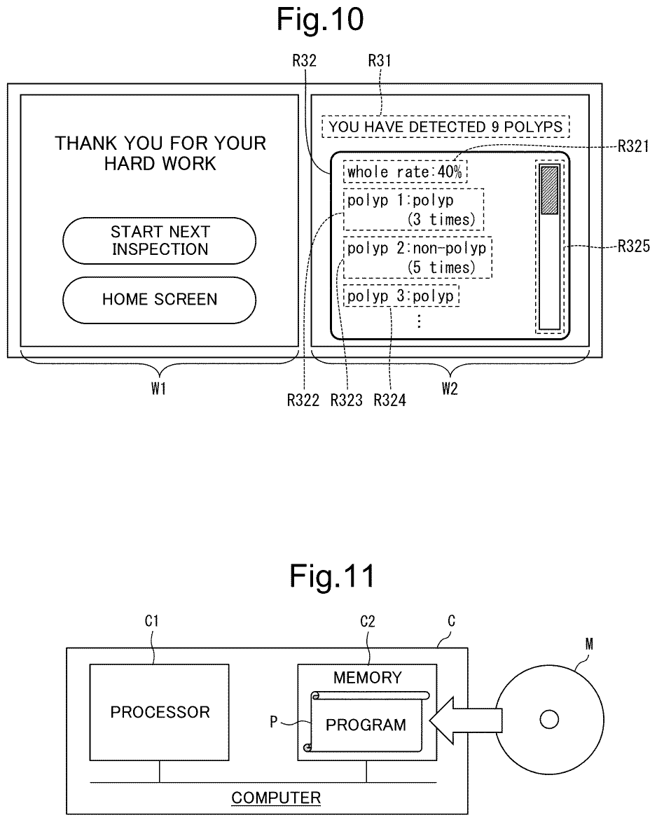

FIG. 10 illustrates an example of a display image generated by the integration unit 15 at a time point when a series of inspections using the endoscope and the information processing device 1A for a certain subject is completed. In the display image illustrated in FIG. 10, a display element “You have detected 9 polyps” indicating the total number of detected polyp candidates (or the number of polyps determined (estimated) to be polyps by the second determination unit) is disposed in the region W2.

The display image includes various types of information indicating the inspection result in the region R32. Specifically, the integrated second type score (integrated inflammation score) calculated by the integration unit 15 is displayed in the region R321, and a display element “polyp 1: polyp (3 times)” indicating that a polyp candidate with a serial number 1 is detected three times and the polyp candidate is determined (estimated) to be a polyp by the second determination unit 16 is disposed in the region R322.

In the region R323, a display element “polyp 2: non-polyp (5 times)” indicating that a polyp candidate with a serial number 2 is detected 5 times and the polyp candidate is determined (estimated) not to be a polyp by the second determination unit 16 is disposed.

In the region R324, a display element “polyp 3: polyp” indicating that a polyp candidate with a serial number 3 is determined (estimated) to be a polyp by the second determination unit 16 is disposed.

A scroll bar is displayed in the region R325 of the display screen, and a viewer of the display screen can view information included in the region R32 while scrolling by operating the scroll bar. Such display control can also be performed by the integration unit 15.

As described above, the integration unit 15 may generate a display image including the second type integrated score and a determination result regarding one or a plurality of polyp candidates, the determination result regarding whether the polyp candidate is a polyp. According to this configuration, it is possible to provide useful information to a medical worker, and thus, it is possible to suitably support the medical worker.

### [Example of Implementation by Software]

The control blocks (in particular, the detection unit 12, the storage control unit 13, the first determination unit 14, the integration unit 15, and the second determination unit 16) of the information processing devices 1 and 1A may be implemented by a logic circuit (hardware) formed in an integrated circuit (IC chip) or the like, or may be implemented by software.

In the latter case, the information processing devices 1 and 1A include a computer that executes a command of a program that is software for achieving each function. The computer includes, for example, at least one processor (control device) and at least one computer-readable recording medium storing the program. Then, in the computer, the processor reads the program from the recording medium and executes the program, whereby the object of the present invention is achieved. As the processor, for example, a central processing unit (CPU) can be used. As the recording medium, a “non-transitory tangible medium”, for example, a tape, a disk, a card, a semiconductor memory, a programmable logic circuit, and the like can be used in addition to a read only memory (ROM) and the like. A random access memory (RAM) or the like for expanding the program may be further provided. The program may be supplied to the computer via an arbitrary transmission medium (communication network, broadcast wave, or the like) capable of transmitting the program. One aspect of the present invention can also be achieved in a form of a data signal embedded in a carrier wave in which the program is embodied by electronic transmission.

### [Supplementary Information 1]

The present invention is not limited to the above-described example embodiments, and various changes can be made without departing from the spirit and the scope as defined by the claims. For example, example embodiments obtained by appropriately combining the technical means disclosed in the above-described example embodiments are also included in the technical scope of the present invention.

### [Supplementary Information 2]

Some or all the above-described example embodiments may be described as follows. However, the present invention is not limited to the following aspects.

### (Supplementary Note 1)

An information processing device including:
an acquisition means for acquiring one or a plurality of endoscopic images;
a detection means for detecting a polyp candidate from the endoscopic image and calculating a first type score indicating a polyp likelihood regarding the polyp candidate;
a storage control means for storing an image of the polyp candidate detected by the detection means and the first type score in a storage unit;
a first determination means for determining whether the polyp candidate detected by the detection means is identical to one or a plurality of other polyp candidates stored in the storage unit; and
an integration means for calculating a first type integrated score by integrating the first type score calculated by the detection means and a first type score related to one or a plurality of the other polyp candidates stored in the storage unit in a case where the first determination means determines that the candidates are identical to each other.

### (Supplementary Note 2)

The information processing device according to Supplementary Note 1, further including
a second determination means for determining the polyp candidate with reference to the first type integrated score.

### (Supplementary Note 3)

The information processing device according to Supplementary Note 2, in which
the second determination means determines whether the polyp candidate is a polyp with reference to the first type integrated score.

### (Supplementary Note 4)

The information processing device according to any one of Supplementary Notes 1 to 3, in which
in a case where the first determination means determines that the candidates are identical to each other, the integration means includes,
in a display image, an endoscopic image acquired by the acquisition means and an image of one or a plurality of the other polyp candidates stored in the storage unit.

### (Supplementary Note 5)

The information processing device according to Supplementary Note 4, in which
the integration means includes the first type integrated score in the display image.

### (Supplementary Note 5)

The information processing device according to Supplementary Note 4 or 5, in which
the integration means includes,
in the display image, a serial number attached to the polyp candidate in a case where the first determination means determines that the candidates are identical to each other.

### (Supplementary Note 7)

The information processing device according to any one of Supplementary Notes 1 to 6, in which
the detection means further calculates a second type score indicating a degree of inflammation from the endoscopic image.

### (Supplementary Note 8)

The information processing device according to Supplementary Note 7, in which
the storage control means stores the second type score in the storage unit.

### (Supplementary Note 9)

The information processing device according to Supplementary Note 8, in which
the integration means calculates a second type integrated score by integrating the second type score calculated by the detection means and a second type score related to one or a plurality of other endoscopic images stored in the storage unit.

### (Supplementary Note 10)

The information processing device according to Supplementary Note 9, in which
the integration means includes the second type integrated score in a display image.

### (Supplementary Note 11)

The information processing device according to Supplementary Note 10, in which
the integration means includes the second type integrated score in the display image in a case where the detection means calculates the second type score a predetermined number of times or more.

### (Supplementary Note 12)

The information processing device according to any one of Supplementary Notes 9 to 11, in which
the integration means
generates a display image including:
the second type integrated score; and
a determination result related to one or a plurality of polyp candidates, the determination result being related to whether the polyp candidate is a polyp.

### (Supplementary Note 13)

An information processing method for causing at least one processor to execute:
acquiring one or a plurality of endoscopic images;
detecting a polyp candidate from the endoscopic image and calculating a first type score indicating a polyp likelihood regarding the polyp candidate;
storing an image of the polyp candidate and the first type score in a storage unit;
determining whether the polyp candidate is identical to one or a plurality of other polyp candidates stored in the storage unit; and
calculating a first type integrated score by integrating the first type score and a first type score related to one or a plurality of the other polyp candidates stored in the storage unit in a case where the candidates are determined to be identical to each other.

### (Supplementary Note 14)

An information processing program for causing a computer to execute:
an acquisition process for acquiring one or a plurality of endoscopic images;
a detection process for detecting a polyp candidate from the endoscopic image and calculating a first type score indicating a polyp likelihood regarding the polyp candidate;
a storage control process for storing an image of the polyp candidate detected by the detection process and the first type score in a storage unit;
a first determination process for determining whether the polyp candidate detected by the detection process is identical to one or a plurality of other polyp candidates stored in the storage unit; and
an integration process for calculating a first type integrated score by integrating the first type score calculated by the detection process and a first type score related to one or a plurality of the other polyp candidates stored in the storage unit in a case where the first determination process determines that the candidates are identical to each other.

### [Supplementary Information 3]

Some or all the above-described example embodiments may be described as follows.

### (Supplementary Note 15)

An information processing device including
at least one processor, in which the processor executes
an acquisition process for acquiring one or a plurality of endoscopic images,
a detection process for detecting a polyp candidate from the endoscopic image and calculating a first type score indicating a polyp likelihood regarding the polyp candidate,
a storage control process for storing an image of the polyp candidate detected by the detection process and the first type score in a storage unit,
a first determination process for determining whether the polyp candidate detected by the detection process is identical to one or a plurality of other polyp candidates stored in the storage unit, and
an integration process for calculating a first type integrated score by integrating the first type score calculated by the detection process and a first type score related to one or a plurality of the other polyp candidates stored in the storage unit in a case where the first determination process determines that the candidates are identical to each other.

This information processing device may further include a memory, and the memory may store a program for causing the processor to execute the acquisition process, the classification process, and the learning process. This program may be recorded in a computer-readable non-transitory tangible recording medium.

### REFERENCE SIGNS LIST

1, 1A information processing device
11 acquisition unit
12 detection unit
13 storage control unit
14 first determination unit
15 integration unit
16 second determination unit
20A storage unit
30A communication unit
40A input/output unit

What is claimed is:

1. An information processing device comprising:
at least one memory storing instructions; and
at least one processor configured to execute the instructions to:
acquire one or a plurality of endoscopic images;
detect a polyp candidate from the endoscopic image;
calculate a first type score indicating a polyp likelihood regarding the polyp candidate;
store an image of the polyp candidate and the first type score in a storage unit;
determine whether the polyp candidate is identical to one or a plurality of other polyp candidates stored in the storage unit; and
calculate a first type integrated score by integrating the first type score and a first type score related to one or a plurality of the other polyp candidates stored in the storage unit when the candidates are determined identical to each other.

2. The information processing device according to claim 1,
wherein the at least one processor is further configured to execute the instructions to:
determine the polyp candidate with reference to the first type integrated score.

3. The information processing device according to claim 2,
wherein the at least one processor is further configured to execute the instructions to:
determine whether the polyp candidate is a polyp with reference to the first type integrated score.

4. The information processing device according to claim 1,
wherein the at least one processor is further configured to execute the instructions to:
include the endoscopic image and an image of one or a plurality of the other polyp candidates in a display image, when the candidates are determined identical to each other.

5. The information processing device according to claim 4,
wherein the at least one processor is further configured to execute the instructions to:
include the first type integrated score in the display image.

6. The information processing device according to claim 4,
wherein the at least one processor is further configured to execute the instructions to:
include a serial number attached to the polyp candidate in the display image, when the candidates are determined identical to each other.

7. The information processing device according to claim 1,
wherein the at least one processor is further configured to execute the instructions to:
calculate a second type score indicating a degree of inflammation from the endoscopic image.

8. The information processing device according to claim 7,
wherein the at least one processor is further configured to execute the instructions to:
store the second type score in the storage unit.

9. The information processing device according to claim 8,
wherein the at least one processor is further configured to execute the instructions to:
calculate a second type integrated score by integrating the second type score and a second type score related to one or a plurality of other endoscopic images stored in the storage unit.

10. The information processing device according to claim 9,
wherein the at least one processor is further configured to execute the instructions to:
include the second type integrated score in a display image.

11. The information processing device according to claim 10,
wherein the at least one processor is further configured to execute the instructions to:
include the second type integrated score in the display image when the second type score is calculated a predetermined number of times or more.

12. The information processing device according to claim 9,
wherein the at least one processor is further configured to execute the instructions to:
generate a display image including,
the second type integrated score; and
a determination result related to one or a plurality of polyp candidates, the determination result being related to whether the polyp candidate is a polyp.

13. An information processing method comprising:
by at least one processor,
acquiring one or a plurality of endoscopic images;
detecting a polyp candidate from the endoscopic image;
calculating a first type score indicating a polyp likelihood regarding the polyp candidate;
storing an image of the polyp candidate and the first type score in a storage unit;
determining whether the polyp candidate is identical to one or a plurality of other polyp candidates stored in the storage unit; and
calculating a first type integrated score by integrating the first type score and a first type score related to one or a plurality of the other polyp candidates stored in the storage unit in a case where the candidates are determined to be identical to each other.

14. A non-transitory recording medium recording a program for causing a computer to execute:
acquiring one or a plurality of endoscopic images;
detecting a polyp candidate from the endoscopic image;
calculating a first type score indicating a polyp likelihood regarding the polyp candidate;
storing an image of the polyp candidate detected by the detection process and the first type score in a storage unit;
determining whether the polyp candidate detected by the detection process is identical to one or a plurality of other polyp candidates stored in the storage unit; and
calculating a first type integrated score by integrating the first type score calculated by the detection process and a first type score related to one or a plurality of the other polyp candidates stored in the storage unit in a case where the first determination process determines that the candidates are identical to each other.

15. The information processing device according to claim 7, wherein the second-type score is calculated by a trained machine learning model and provided to support physician decision making.
